(19) United States
(12) Patent Application Publication
(10) Pub. No.: US 2019/0290500 A1
(43) Pub. Date: Sep. 26, 2019

(54) Intelligent diaper

(71) Applicants: Zuo Chen, Shenzhen (CN); Xiaojian Gao, Shenzhen (CN)

(72) Inventors: Zuo Chen, Shenzhen (CN); Xiaojian Gao, Shenzhen (CN)

(21) Appl. No.: 16/436,946

(22) Filed: Jun. 11, 2019

(51) Int. Cl. A61F 13/42

(52) U.S. Cl. CPC A61F 13/42; A61F 2013/8473; A61F 2013/422

(57) ABSTRACT

An intelligent diaper includes an inner layer configured to be substantially liquid-pervious; a transparent outer layer configured to be substantially liquid-impervious; a middle layer disposed between the inner layer and the transparent outer layer and configured to absorb liquid from a diaper wearer's body; a core member disposed between the transparent outer layer and the middle layer, and the core member including an identification zone having a 2D barcode, a color calibration zone, and a test zone having two spaced test paper strips; urine indication strips disposed between the transparent outer layer and the middle layer and being adjacent to the core member; a negative comparison zone having two spaced negative color comparison cards; and a positive comparison zone having two spaced positive color comparison cards. Color of the urine indication strips changes in response to expelled waste.

### BACKGROUND OF THE INVENTION

### 1. Field of the Invention

The invention relates to diapers for infants and more particularly to an intelligent diaper capable of determining whether a diaper wearer has expelled the waste or nor, and the diaper wearer has contracted a disease or not.

### 2. Description of Related Art

Feces and urine are regularly expelled out of humans. Also, feces and urine can be tested for determining whether a person has diseases including kidney disease, liver disease, diabetes, etc. Also, feces and urine can be tested for monitoring medication, healing, etc.

It is not easy to collect feces and urine of an infant. Further, conventional diapers do not allow a parent to visually observe that the diaper is wet. It is typical for the parent to touch the diaper to determine whether the diaper is wet or not. Thus, a timely change of diaper is not possible. It can result in skin problems around the area covered by the diaper.

Thus, the need for improvement still exists.

### SUMMARY OF THE INVENTION

The invention has been made in an effort to solve the problems of the conventional art by providing an intelligent diaper having novel and nonobvious characteristics.

To achieve above and other objects of the invention, the invention provides an intelligent diaper comprising an inner layer configured to be substantially liquid-pervious; a transparent outer layer configured to be substantially liquid-impervious; a middle layer disposed between the inner layer and the transparent outer layer and configured to absorb liquid from a diaper wearer's body; a core member disposed between the transparent outer layer and the middle layer, the core member corresponding to the crotch of the diaper wearer, and the core member including an identification zone having a 2D barcode, a color calibration zone, and a test zone having two spaced test paper strips; a plurality of urine indication strips disposed between the transparent outer layer and the middle layer and being adjacent to the core member, the urine indication strips corresponding to the crotch of the diaper wearer; a negative comparison zone disposed in a first predetermined position and having two spaced negative color comparison cards; and a positive comparison zone disposed in a second predetermined position and having two spaced positive color comparison cards.

Preferably, the urine indication strips are provided on front and rear portions of the intelligent diaper.

Preferably, the urine indication strips are spaced apart one another.

Preferably, the first and second predetermined positions are around the core member and between the transparent outer layer and the middle layer.

Preferably, the first and second predetermined positions are around the core member and correspond to the transparent outer layer.

Preferably, the positive and negative color comparison cards correspond to the test paper strips respectively.

Preferably, the core member further comprises two identification zones having a plurality of labels corresponding to elements of each test paper strip respectively.

Preferably, further comprises a layer of cushion provided on the inner layer corresponding to the hip of the diaper wearer.

The invention has the following advantageous effects in comparison with the prior art: Improved liquid-pervious, quickly and correctly determining whether a diaper wearer is healthy, improved health management, and decrease of the times of going to hospital.

The above and other objects, features and advantages of the invention will become apparent from the following detailed description taken with the accompanying drawings.

### BRIEF DESCRIPTION OF THE DRAWINGS

FIG. 1 is a longitudinal sectional view of an intelligent diaper according to a first preferred embodiment of the invention;

FIG. 2 schematically depicts a first configuration of the core member;

FIG. 3 schematically depicts a second configuration of the core member; and

FIG. 4 is a longitudinal sectional view of an intelligent diaper according to a second preferred embodiment of the invention.

### DETAILED DESCRIPTION OF THE INVENTION

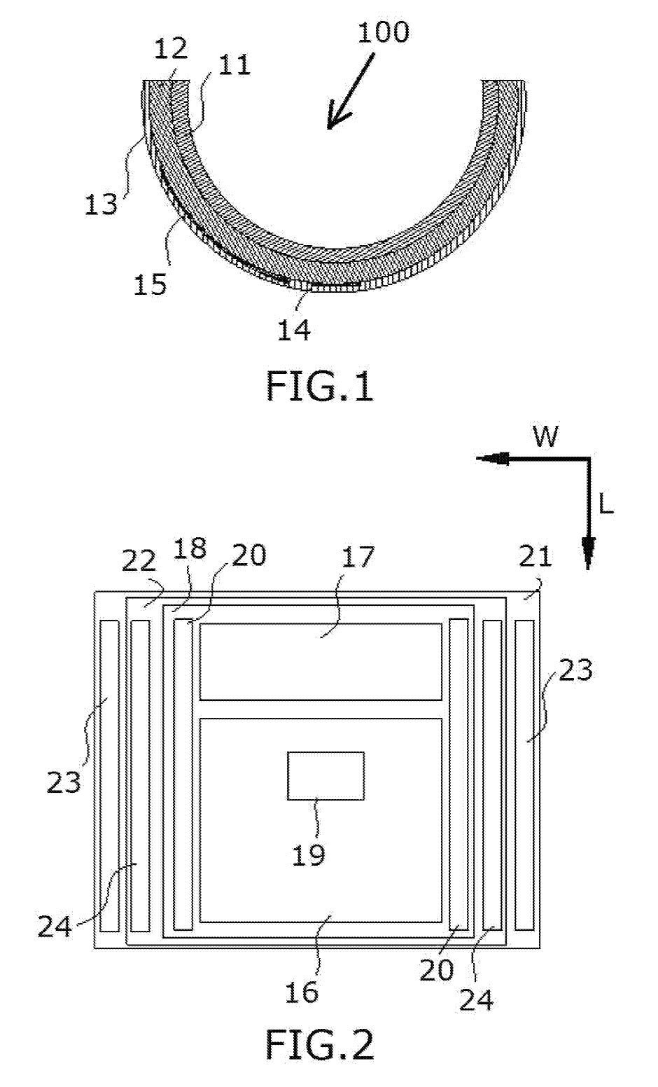

Referring to FIGS. 1 to 3, an intelligent diaper 100 in accordance with a first preferred embodiment of the invention comprises an inner layer 11 configured to be substantially liquid-pervious, a transparent outer layer 13 configured to be substantially liquid-impervious, a middle layer 12 configured to absorb liquid from a diaper wearer's body and disposed between the inner layer 11 and the transparent outer layer 13, a core member 14 at a crotch in the transparent outer layer 13, and a plurality of urine indication strips 15 at the crotch in the transparent outer layer 13 and adjacent to the core member 14. The core member 14 includes an identification zone 16 having a 2D barcode 19, a color calibration zone 17, and a test zone 18 having two spaced test paper strips 20. Around the core member 14, there are provided a negative comparison zone 22 having two spaced negative color comparison cards 24. Around the negative comparison zone 22, there are provided a positive comparison zone 21 having two spaced positive color comparison cards 23.

After the wearer has expel feces and/or urine, the waste passes through the inner layer 11 and the middle layer 12 into the urine indication strips 15. As a result, color of the urine indication strips 15 changes due to chemical reaction of the urine indication strips 15 and the waste. Thus, a parent may determine whether the infant has expelled waste or not by seeing the urine indication strips 15. If the color of the urine indication strips 15 changes, the parent may change the intelligent diaper 100. Also, the waste passes through the inner layer 11 and the middle layer 12 into the core member 14. Further, the waste contacts the test paper strips 20 of the test zone 18 of the core member 14. As a result, color of the test paper strips 20 changes due to chemical reaction of the test paper strips 20 and the waste. Thus, a parent may determine whether the infant has expelled waste or not by seeing the test paper strip 20 through the transparent outer layer 13.

Further, the parent may compare color of the test paper strip 20 with colors of the positive comparison zone 21 and the negative comparison zone 22 confirm whether the infant has contracted a disease or not. In addition and/or alternatively, the parent may use a cellular phone to scan the 2D barcode 19 in the identification zone 16 to know medical history of the infant because health examination and other health information are stored in the 2D barcode 19.

All of materials of the inner layer 11, the middle layer 12 and the transparent outer layer 13 are known in the art such as plastic. Material of the urine indication strips 15 is any one in the art that may react with urine to cause color change. It is possible of knowing disease that the infant has contracted by seeing color change of the negative color comparison card 24 and/or color change of the positive color comparison card 23. A well known color calibration card is provided in the color calibration zone 17. The urine indication strips 15 are provided on a front portion (e.g., portion contacting the abdomen) of the intelligent diaper 100 and a rear portion (e.g., portion contacting the hips) of the intelligent diaper 100. The urine indication strip 15 may be provided in equal spacing, gradually increased spacing or gradually decreased spacing. As shown in FIG. 2 specifically, in a first configuration, the color calibration zone 17 and the identification zone 16 are spaced apart along a lengthwise direction, indicated by L and in contrast to a widthwise direction indicated by W, of an intermediate portion of the core member 14. The test zone 18 is provided around both the color calibration zone 17 and the identification zone 16. The negative comparison zone 22 is provided around the test zone 18. The positive comparison zone 21 is provided around the negative comparison zone 22. In case of color change of the test paper strip 20, color of the test paper strip 20 is compared with color of the negative comparison zone 22. If there are differences between the colors, it is determined that there is something wrong with the infant. Further, the parent may use a cellular phone to scan the 2D barcode 19 in the identification zone 16 to know details of the disease contracted by the infant. To the contrary, if there is no difference between the colors, it is determined that the infant is healthy. The determination is quick and correct. Further, it can improve health management and decrease the times of going to hospital.

The positive comparison zone 21 and the negative comparison zone 22 are around the core member 14 between the middle layer 12 and the transparent outer layer 13. In other embodiments, the positive comparison zone 21 and the negative comparison zone 22 are around the core member 14 in the transparent outer layer 13.

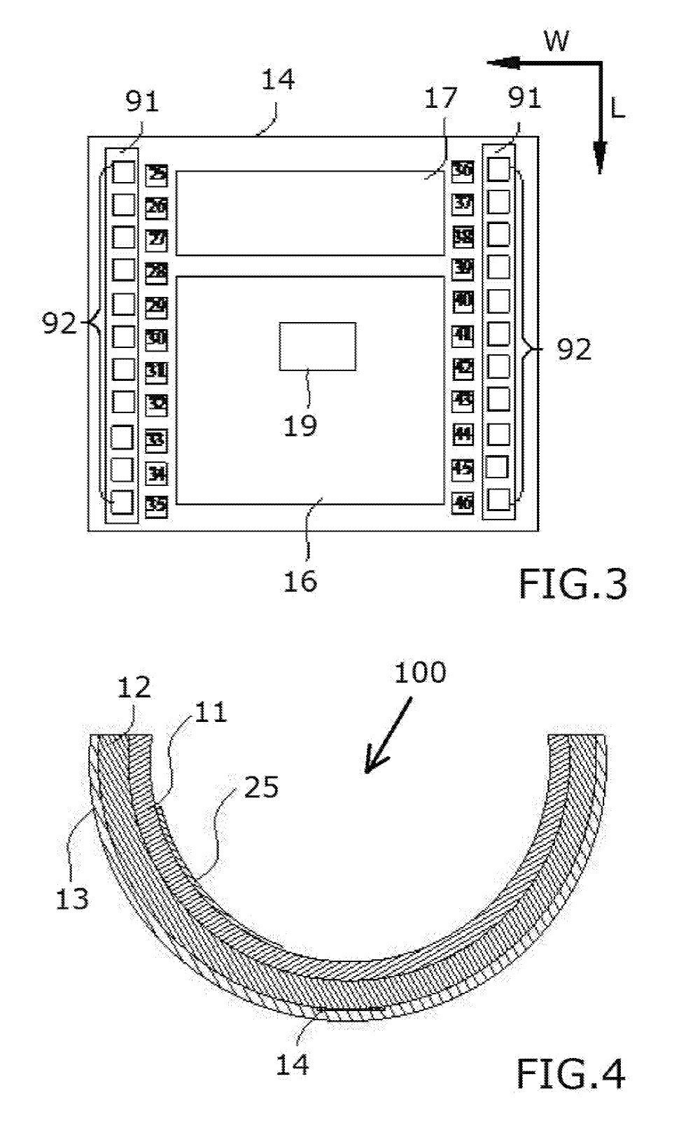

As shown in FIG. 3 specifically, in a second configuration, the core member 14 comprises two identification zones 91 having a plurality of labels 92 corresponding to elements of the test paper strip 20 respectively. The labels 92 are Arabic numbers, Roman numbers or English alphabets. By utilizing the labels 92, it is possible of quickly identifying corresponding health information represented by the 2D barcode 19.

Preferably, size of the core member 14 is 50-100 mm×50-100 mm.

Referring to FIG. 4, an intelligent diaper 100 in accordance with a second preferred embodiment of the invention is shown. The characteristics of the second preferred embodiment are substantially the same as that of the first preferred embodiment except the following: A layer of cushion 25 is provided on the inner layer 11 corresponding to the hip so as to provide a degree of comfort to the diaper wearer. Material of the layer of cushion 25 is well known in the art. Further, thickness of the layer of cushion 25 can be chosen to be appropriate. Further, a plurality of grooves (not shown) having a depth of 1-3 mm are provided on the surface of the layer of cushion 25 for providing liquid-pervious characteristic.

While the intelligent diaper is suitable for infants aged no more than two years, it is also applicable to toddlers, pregnant women, senior citizens, diabetes patients, or kidney patients.

While the invention has been described in terms of preferred embodiments, those skilled in the art will recognize that the invention can be practiced with modifications within the spirit and scope of the appended claims.

What is claimed is:

1. An intelligent diaper comprising:
an inner layer configured to be substantially liquid-pervious;
a transparent outer layer configured to be substantially liquid-impervious;
a middle layer disposed between the inner layer and the transparent outer layer and configured to absorb liquid from a diaper wearer's body;
a core member disposed between the transparent outer layer and the middle layer, the core member corresponding to the crotch of the diaper wearer, and the core member including an identification zone having a 2D barcode, a color calibration zone, and a test zone having two spaced test paper strips;
a plurality of urine indication strips disposed between the transparent outer layer and the middle layer and being adjacent to the core member, the urine indication strips corresponding to the crotch of the diaper wearer;
a negative comparison zone disposed in a first predetermined position and having two spaced negative color comparison cards; and
a positive comparison zone disposed in a second predetermined position and having two spaced positive color comparison cards.

2. The intelligent diaper of claim 1, wherein the urine indication strips are disposed on front and rear portions of the intelligent diaper.

3. The intelligent diaper of claim 1, wherein the urine indication strips are spaced apart one another.

4. The intelligent diaper of claim 1, wherein the first and second predetermined positions are around the core member and between the transparent outer layer and the middle layer.

5. The intelligent diaper of claim 1, wherein the first and second predetermined positions are around the core member and correspond to the transparent outer layer.

6. The intelligent diaper of claim 1, wherein the positive and negative color comparison cards correspond to the test paper strips respectively.

7. The intelligent diaper of claim 1, wherein the core member further comprises two identification zones having a plurality of labels corresponding to elements of each test paper strip respectively.

8. The intelligent diaper of claim 1, further comprising a layer of cushion disposed on the inner layer corresponding to the hip of the diaper wearer.
